(19) United States
(12) Patent Application Publication
(10) Pub. No.: US 2017/0156675 A1
(43) Pub. Date: Jun. 8, 2017

(54) Detection method of body tumble event, electronic device and mobile terminal

(71) Applicants: LE HOLDINGS (BEIJING) CO., LTD., Beijing (CN); LEMOBILE INFORMATION TECHNOLOGY (BEIJING) CO., LTD., Beijing (CN)

(72) Inventor: Peng SUN, Beijing (CN)

(21) Appl. No.: 15/242,067

(22) Filed: Aug. 19, 2016

Related U.S. Application Data
(63) Continuation of application No. PCT/CN2016/088733, filed on Jul. 5, 2016.

(30) Foreign Application Priority Data
Dec. 8, 2015 (CN) 2015108969893

(51) Int. Cl. A61B 5/00; H04W 64/00; A61B 5/11; H04L 29/08

(52) U.S. Cl. CPC A61B 5/6898; H04L 67/12; H04L 67/26; H04W 64/006; A61B 5/1117; A61B 5/7282; A61B 5/747; H04W 88/02

(57) ABSTRACT

Embodiments of the disclosure disclose a detection method of a body tumble event, electronic device and a mobile terminal. The method comprises: acquiring acceleration values from an acceleration sensor at a mobile terminal; computing whether acceleration suddenly increases and maintains at zero within a preset period after suddenly increasing, according to the acquired acceleration values; and if YES, determining that the body tumble event occurs, and sending warning information to an emergency contact person preset in the mobile terminal, wherein the warning information carries current position information.

### CROSS REFERENCE TO RELATED APPLICATIONS

The present application is a continuation of PCT application which has an application number of PCT/CN2016/088733 and was filed on Jul. 5, 2016. This application is based upon and claims priority to Chinese Patent Application NO. 2015108969893, titled “Detection Method and Device of Body Tumble Event, and Mobile Terminal”, filed with the Chinese Patent Office on Dec. 8, 2015, the entire contents of which are incorporated herein by reference.

### TECHNICAL FIELD

The application relates to the field of mobile application technology, and particularly to a detection method of a body tumble event, electronic device and a mobile terminal.

### BACKGROUND

In our daily life, we can often read news that a person tumbled but no passerby dared to give assistance, and sometimes a regret result would even be caused for missing precious rescue time. On the one hand, we should avoid occurrence of such things by moral condemnation and legal sanction; on the other hand, we are also thinking about how to solve such problems by adopting a technical means.

At present, mobile terminals (e.g. cell phones) have become belongings carried along with people most frequently. In the existing technology, it is still impossible to carry out a solution of detecting a tumble event of a body by using a mobile terminal.

### SUMMARY

In view of this, the embodiments of the present application aim to provide a detection solution of a body tumble event, so as to solve timely detection of a tumble event occurring to a body.

In the first aspect of the disclosure, an embodiment of the present application provides a detection method of a body tumble event. The method comprises: acquiring acceleration values from an acceleration sensor at a mobile terminal; computing whether acceleration suddenly increases and maintains at zero within a preset period after suddenly increasing, according to the acquired acceleration values; and if YES, determining that the body tumble event occurs, and sending warning information to an emergency contact person preset in the mobile terminal, wherein the warning information carries current position information.

In the second aspect of the disclosure, an embodiment of the present application provides an electronic device. The electronic device comprises: at least one processor; and a memory communicably connected with the at least one processor for storing instructions executable by the at least one processor, wherein execution of the instructions by the at least one processor causes the at least one processor to: acquire acceleration values from an acceleration sensor at a mobile terminal; compute whether acceleration suddenly increases and maintains at zero within a preset period after suddenly increasing, according to acquired acceleration values; and if YES, determine that the body tumble event occurs, and send warning information to an emergency contact person preset in the mobile terminal, wherein the warning information carries current position information.

In the third aspect of the disclosure, an embodiment of the present application provides a non-transitory computer-readable storage medium storing executable instructions that, when executed by an electronic device, cause the electronic device to: acquire acceleration values from an acceleration sensor at a mobile terminal; compute whether acceleration suddenly increases and maintains at zero within a preset period after suddenly increasing, according to acquired acceleration values; and if YES, determine that the body tumble event occurs, and send warning information to an emergency contact person preset in the mobile terminal, wherein the warning information carries current position information.

In the fourth aspect of the disclosure, an embodiment of the present application provides a mobile terminal. The mobile terminal may comprise: a positioning module for acquiring geographical position information of the mobile terminal; an acceleration sensor for collecting acceleration values of the mobile terminal; a sensor co-processor for acquiring the acceleration values from the acceleration sensor at the mobile terminal, and computing whether acceleration suddenly increases and maintains at zero within a preset period after suddenly increasing, according to the acquired acceleration values, and if YES, determining that the body tumble event occurs, and sending a notification message of the body tumble event to an application processor; the application processor for acquiring current position information from the positioning module upon receipt of the notification message of the body tumble event, sending warning information carrying the current position information to a modem; and the modem for sending the warning information to an emergency contact person preset in the mobile terminal.

An embodiment of the present application provides a detection method of a body tumble event, which persistently detects a body tumble event and, once the body tumble event occurs, can send current position information to an emergency contact person preset in a mobile terminal, thereby making it possible to enable a person encountering a danger to be provided with assistance timely, so as to reduce damages to a body.

### BRIEF DESCRIPTION OF THE DRAWINGS

One or more embodiments are illustrated by way of example, and not by limitation, in the figures of the accompanying drawings, wherein elements having the same reference numeral designations represent like elements throughout. The drawings are not to scale, unless otherwise disclosed.

FIG. 1 illustrates a flow chart of a detection method of a body tumble event in accordance with one embodiment of the application;

FIG. 2 illustrates a flow chart of a method for detecting acceleration corresponding to a body tumble event in accordance with one embodiment of the application;

FIG. 3 illustrates a block diagram of a detection device of a body tumble event in accordance with one embodiment of the application;

FIG. 4 illustrates a schematic view of constitution of a mobile terminal, which can be used for detecting a body tumble event, in accordance with one embodiment of the application;

FIG. 5 illustrates a hardware structure of an electronic device according to an embodiment of the present application.

### DETAILED DESCRIPTION

Hereinafter, exemplary embodiments of the disclosure will be described in more detail with reference to the accompanying drawings. Although the exemplary embodiments of the disclosure are shown in the accompanying drawings, it should be understood that the disclosure can be realized in various forms, which should not be limited by the embodiments disclosed herein. Instead, the embodiments are provided to enable more thorough understanding to the disclosure, and to enable a person skilled in the art to get acquaintance to the whole scope of the disclosure.

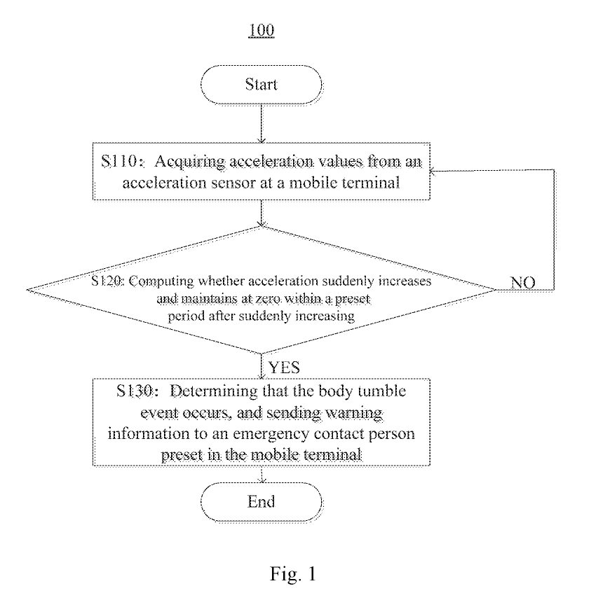

Referring to FIG. 1, FIG. 1 illustrates a flow chart of a detection method 100 of a body tumble event in accordance with one embodiment of the disclosure.

In step S110, acceleration values are acquired from an acceleration sensor at a mobile terminal.

In step S120, it is computed whether acceleration suddenly increases and maintains at zero within a preset period after suddenly increasing, according to the acquired acceleration values.

On the one hand, acceleration of a body will observe the following mode upon occurrence of a tumble event to the body: the acceleration value suddenly increases in the sudden tumble process of the body, the acceleration value will change to zero after the body tumbles to the ground, and the acceleration value will maintain at zero and persistently unchanged if the body is in a tumble state all the time. Hence, whether a body tumble event occurs can be detected from a change condition of the acceleration value of the body. On the one hand, the mobile terminal is worn along with people, and information on its movement path, movement speed, acceleration and the like is synchronous with information on a movement path, movement speed, acceleration and the like of the body, so an acceleration change of the body can be detected by detecting an acceleration change of the mobile terminal.

In step S130, if a computation result in step S120 is YES, it is determined that the body tumble event occurs, and warning information is sent to an emergency contact person preset in the mobile terminal, wherein the warning information carries current position information. In this way, the emergency contact person can know the body tumble event timely, and can know a geographical position where the tumble event occurs.

In one embodiment, the warning information further comprises current time information of the mobile terminal, so the emergency contact person can know accurate time when the tumble event occurs.

In one embodiment, the warning information can be sent to the emergency contact person preset in the mobile terminal by a short message, automatic voice, or any other proper manner that can be supported by the mobile terminal.

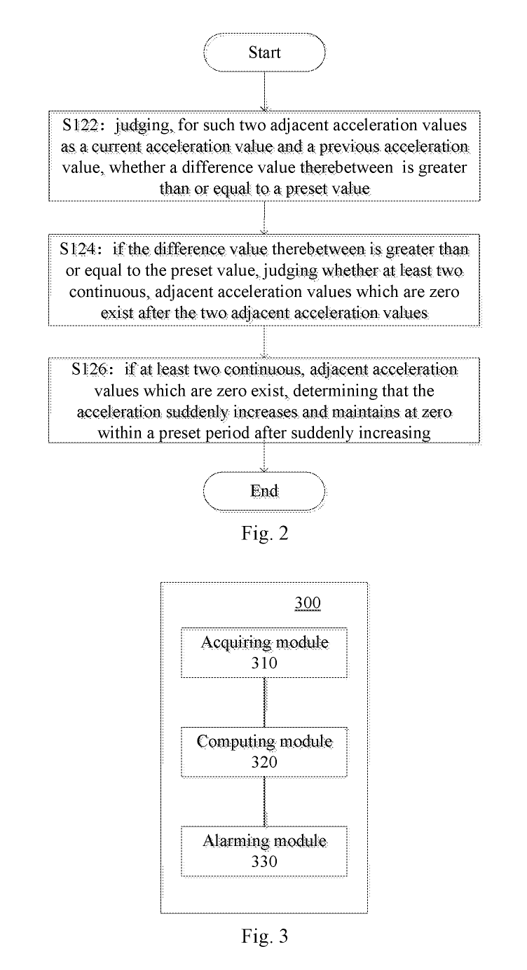

Referring to FIG. 2, FIG. 2 illustrates a flow chart of an exemplary implementation method of step S120 in the method 100 as shown in FIG. 1. The method can be used for detecting acceleration corresponding to a body tumble event.

In step S122, it is judged, for such two adjacent acceleration values as a current acceleration value and a previous acceleration value, whether a difference value between the two adjacent acceleration values is greater than or equal to a preset value.

In step S124, if the difference value between the two adjacent acceleration values is greater than or equal to the preset value, it is judged whether at least two continuous, adjacent acceleration values which are zero exist after the two adjacent acceleration values.

In step S126, if at least two continuous, adjacent acceleration values which are zero exist after the two adjacent acceleration values, it is determined that the acceleration suddenly increases and maintains at zero within a preset period after suddenly increasing.

In one embodiment, a contrast curve can be drawn according to the foregoing change mode of the acceleration of the body upon occurrence of the tumble event. The contrast curve exhibits a spike dropping to zero proximately in a straight line after sudden rising, and maintains constant for a duration at zero value. The detection of the acceleration corresponding to the body tumble event may also be: detecting and recording body acceleration values within the latest preset period of time, and contrasting the recorded acceleration values within the preset period of time with the foregoing contrast curve, and determining that the body tumble event occurs if the recorded acceleration values are in compliance with the foregoing contrast curve.

In an embodiment of the present application, a warning message can be sent to the emergency contact person for more than once, so as to prevent the emergency contact person from neglecting viewing the warning information for being busy when receiving the warning message (e.g. a short message) for a first time or prevent such a possibility as that the emergency contact person fails to hear prompt sound of a new warning short message coming for the first time. An interval period of re-sending the warning message can be set; for example a timer can be started in the mobile terminal. In this way, the emergency contact person is ensured as far as possible to timely view the warning information.

According to an embodiment of the disclosure, as shown in FIG. 1, if the computation result in step S120 is NO, return to step S110, and enter a continuative detection state.

FIG. 3 illustrates a block diagram of a detection device 300 of a body tumble event in accordance with one embodiment of the disclosure. The detection device 300 comprises: an acquiring module 310 for acquiring acceleration values from an acceleration sensor at a mobile terminal; a computing module 320 for computing whether acceleration suddenly increases and maintains at zero within a preset period after suddenly increasing, according to the acquired acceleration values; and a warning module 330 for, if computed acceleration suddenly increases and maintains at zero within a preset period after suddenly increasing, determining that the body tumble event occurs, and sending warning information to an emergency contact person preset in the mobile terminal, wherein the warning information carries current position information.

It should be understood that each module disclosed in the device 300 corresponds to each step in the method 100 as described with reference to FIG. 1. Accordingly, the foregoing operations and features described in regard to FIG. 1 also apply to the device 300 and the modules included therein, which will not be described redundantly herein.

As an example, in one embodiment, the computing module 320 may further comprise: a first judging module for, for such two adjacent acceleration values as a current acceleration value and a previous acceleration value, judging whether a difference value between the two adjacent acceleration values is greater than or equal to a preset value; a second judging module for, if the difference value between the two adjacent acceleration values is greater than or equal to the preset value, judging whether at least two continuous, adjacent acceleration values which are zero exist after the two adjacent acceleration values; and a determining module for, if at least two continuous, adjacent acceleration values which are zero exist after the two adjacent acceleration values, determining that the acceleration suddenly increases and maintains at zero within a preset period after suddenly increasing.

It should be understood that the detection method 100 and the detection device 300 of a body tumble event as proposed by an embodiment of the disclosure can be realized as software, hardware, or a combination of software and hardware. The hardware can be realized by an Application Specific Integrated Circuit (ASIC), a Digital Signal Processor (DSP), a Programmable Logic Device (PLD), a Field Programmable Gate Array (FPGA), a processor, a controller, a microprocessor, other electronic units or combinations thereof which are designed to implement the above functions. The software can be realized by modules implementing the above functions. The software can be stored in a storage unit and be run by a processor. As the storage unit or the processor, various units well-known to a person skilled in the art can be used.

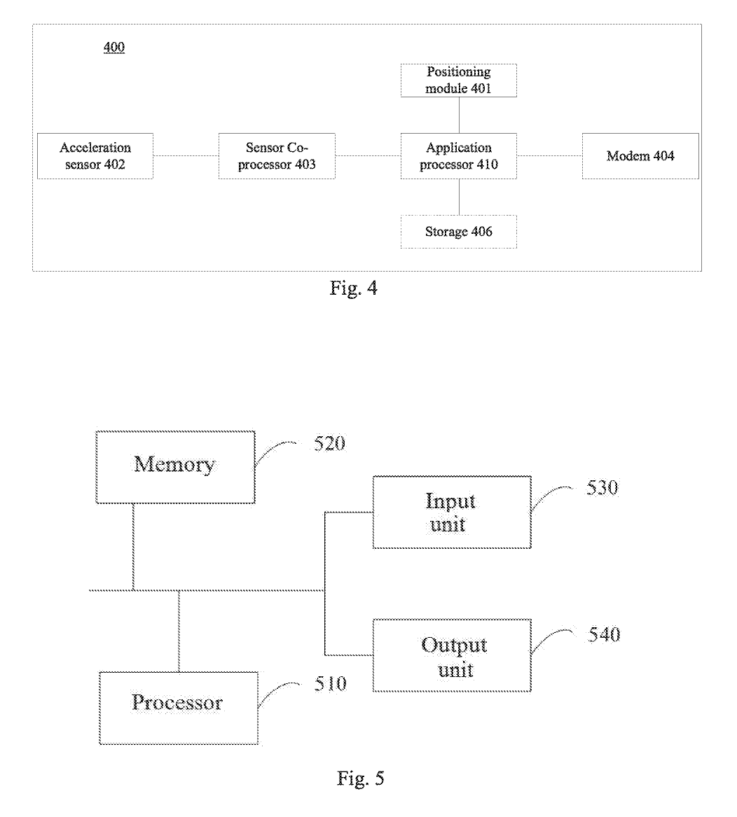

FIG. 4 illustrates a schematic view of constitution of a mobile terminal 400, which can be used for detecting a body tumble event, in accordance with an embodiment of the present application. As shown in FIG. 4, the mobile terminal 400 may comprise an application processor 410, a positioning module 401, an acceleration sensor 402, a sensor co-processor 403 and a modem 404.

The positioning module 401 is used for acquiring geographical position information of the mobile terminal. The acceleration sensor 402 is used for collecting acceleration values of the mobile terminal. The sensor co-processor 403 is connected with the acceleration sensor 402, for acquiring the acceleration values from the acceleration sensor 402 at the mobile terminal, and computing whether acceleration suddenly increases and maintains at zero within a preset period after suddenly increasing, according to the acquired acceleration values, and if YES, determining that the body tumble event occurs, and sending a notification message of the body tumble event to an application processor. The present application processor 410 is connected with the sensor co-processor 403, for acquiring current position information from the positioning module 401 upon receipt of the notification message of the body tumble event, and sending warning information carrying the current position information to the modem. The modem 404 is connected with the application processor 410, for sending the warning information to an emergency contact person preset in the mobile terminal.

As shown in FIG. 4, the mobile terminal 400 may further comprise an exemplary storage 406. It should be understood that the respective components of the mobile terminal 400 as shown in FIG. 4 are only exemplary. To avoid the embodiments of the disclosure from being obscured, other components such as the storage 406 which are possibly included in the mobile terminal 400 are not shown. For example, the mobile terminal 400 includes a display, an input key, various communication interfaces and antennas and the like.

In one embodiment, the sensor co-processor 403 may use a Sensor Hub DSP. Sensor Hub DSP is an always-on, low power consumption chip equipped for almost each mobile terminal, which can satisfy the requirement for persistent detection of the acceleration values of the mobile terminal while reducing the operation burden of a main processor of the mobile terminal.

In one embodiment, the aforementioned detection method 100 of a body tumble event and the aforementioned detection device 300 of a body tumble event according to the embodiment of the disclosure can be implemented at the sensor co-processor 403, without needing to add an additional component or software module in the mobile terminal. However, it should be understood that the aforementioned detection method 100 and the aforementioned detection device 300 can also be implemented at other components than the sensor co-processor 403 in the mobile terminal, for example can be implemented at the acceleration sensor or implemented as a newly added component.

The mobile terminal 400 for example may be a cell phone, a tablet computer, a portable computer, an intelligent watch, a Bluetooth headset or other proper portable devices.

Further, an embodiment of the present application further provides a non-transitory computer-readable storage medium storing executable instructions, which can be executed by an electronic device to perform any methods for detecting a body tumble event mentioned by embodiments of the present application.

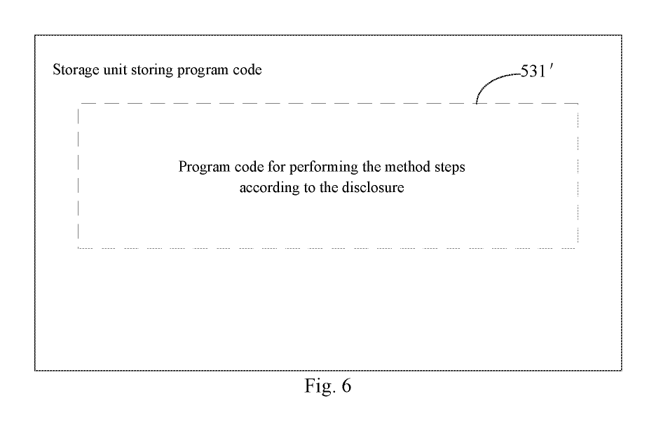

Further, an embodiment of the present application further provides an electronic device. FIG. 5 is a block diagram of an electronic device according to an embodiment of the present application. As shown in FIG. 5, the device includes:

one or more processors 510 and memory 520. A processor 510 is showed in FIG. 5 for an example.

The device can also include: input unit 530 and output unit 540.

Processor 510, memory 520, input unit 530 and output unit 540 can be connected by BUS or other methods, and BUS connecting is showed in FIG. 5 for an example.

Memory 520 can be used for storing non-transitory software program, non-transitory computer executable program and modules as a non-transitory computer-readable storage medium, such as corresponding program instructions/modules for the methods for detecting a body tumble event mentioned by embodiments of the present application (such as shown in FIG. 3, acquiring module 310, computing module 320 and warning module 330). Processor 510 performs kinds of functions and detecting a body tumble event of the electronic device by executing non-transitory software program, instructions and modules which are stored in memory 520, thereby realizes the methods for detecting a body tumble event mentioned by embodiments of the present application.

Memory 520 can include program storage area and data storage area, thereby the operating system and applications required by at least one function can be stored in program storage area and data created by using the device for detecting a body tumble event can be stored in data storage area. Furthermore, memory 520 can include high speed Random-access memory (RAM) or non-volatile memory such as magnetic disk storage device, flash memory device or other non-volatile solid state storage devices. In some embodiments, memory 520 can include long-distance setup memories relative to processor 510, which can communicate with the device for detecting a body tumble event by networks. The examples of said networks are including but not limited to Internet, Intranet, LAN, mobile Internet and their combinations.

Input unit 530 can be used to receive inputted number, character information and key signals causing user configures and function controls of the device for detecting a body tumble event. Output unit 540 can include a display screen or a display device.

The said module or modules are stored in memory 520 and perform the methods for detecting a body tumble event when executed by one or more processors 510.

The said device can reach the corresponding advantages by including the function modules or performing the methods provided by embodiments of the present application. Those methods can be referenced for technical details which may not be completely described in this embodiment.

Electronic devices in embodiments of the present application can be existences with different types, which are including but not limited to:

(1) Mobile Internet devices: devices with mobile communication functions and providing voice or data communication services, which include smartphones (e.g. iPhone), multimedia phones, feature phones and low-cost phones.

(2) Super mobile personal computing devices: devices belong to category of personal computers but mobile internet function is provided, which include PAD, MID and UMPC devices, e.g. iPad.

(3) Portable recreational devices: devices with multimedia displaying or playing functions, which include audio or video players, handheld game players, e-book readers, intelligent toys and vehicle navigation devices.

(4) Servers: devices with computing functions, which are constructed by processors, hard disks, memories, system BUS, etc. For providing services with high reliabilities, servers always have higher requirements in processing ability, stability, reliability, security, expandability, manageability, etc., although they have a similar architecture with common computers.

(5) Other electronic devices with data interacting functions.

The embodiments of devices are described above only for illustrative purposes. Units described as separated portions may be or may not be physically separated, and the portions shown as respective units may be or may not be physical units, i.e., the portions may be located at one place, or may be distributed over a plurality of network units. A part or whole of the modules may be selected to realize the objectives of the embodiments of the present application according to actual requirements.

In view of the above descriptions of embodiments, those skilled in this art can well understand that the embodiments can be realized by software plus necessary hardware platform, or may be realized by hardware. Based on such understanding, it can be seen that the essence of the technical solutions in the present application (that is, the part making contributions over prior arts) may be embodied as software products. The computer software products may be stored in a computer readable storage medium including instructions, such as ROM/RAM, a magnetic disk, an optical disk, to enable a computer device (for example, a personal computer, a server or a network device, and so on) to perform the methods of all or a part of the embodiments.

It shall be noted that the above embodiments are disclosed to explain technical solutions of the present application, but not for limiting purposes. While the present application has been described in detail with reference to the above embodiments, those skilled in this art shall understand that the technical solutions in the above embodiments can be modified, or a part of technical features can be equivalently substituted, and such modifications or substitutions will not make the essence of the technical solutions depart from the spirit or scope of the technical solutions of various embodiments in the present application.

1-11. (canceled)

12. A detection method of a body tumble event, comprising:
acquiring acceleration values from an acceleration sensor at a mobile terminal;
computing whether acceleration suddenly increases and maintains at zero within a preset period after suddenly increasing, according to acquired acceleration values; and
if YES, determining that the body tumble event occurs, and sending warning information to an emergency contact person preset in the mobile terminal, wherein the warning information carries current position information.

13. The method according to claim 12, wherein the warning information further comprises current time information.

14. The method according to claim 12, wherein the sending warning information to an emergency contact person preset in the mobile terminal is:
sending warning information to an emergency contact person preset in the mobile terminal by a short message or automatic voice.

15. The method according to claim 13, wherein the sending warning information to an emergency contact person preset in the mobile terminal is:
sending warning information to an emergency contact person preset in the mobile terminal by a short message or automatic voice.

16. The method according to claim 12, wherein the computing whether acceleration suddenly increases and maintains at zero within a preset period after suddenly increasing, according to the acquired acceleration values, comprises:
judging, for such two adjacent acceleration values as a current acceleration value and a previous acceleration value, whether a difference value between the two adjacent acceleration values is greater than or equal to a preset value;
if the difference value between the two adjacent acceleration values is greater than or equal to the preset value, judging whether at least two continuous, adjacent acceleration values which are zero exist after the two adjacent acceleration values; and
if at least two continuous, adjacent acceleration values which are zero exist after the two adjacent acceleration values, determining that the acceleration suddenly increases and maintains at zero within a preset period after suddenly increasing.

17. The method according to claim 13, wherein the computing whether acceleration suddenly increases and maintains at zero within a preset period after suddenly increasing, according to the acquired acceleration values, comprises:
judging, for such two adjacent acceleration values as a current acceleration value and a previous acceleration value, whether a difference value between the two adjacent acceleration values is greater than or equal to a preset value;
if the difference value between the two adjacent acceleration values is greater than or equal to the preset value, judging whether at least two continuous, adjacent acceleration values which are zero exist after the two adjacent acceleration values; and
if at least two continuous, adjacent acceleration values which are zero exist after the two adjacent acceleration values, determining that the acceleration suddenly increases and maintains at zero within a preset period after suddenly increasing.

18. The method according to claim 12, wherein the sending warning information to an emergency contact person preset in the mobile terminal is:
starting a preset timer after sending warning information for a first time to the emergency contact person preset in the mobile terminal; and
when the timer expires, sending the warning information for a second time to the emergency contact person preset in the mobile terminal.

19. The method according to claim 13, wherein the sending warning information to an emergency contact person preset in the mobile terminal is:
starting a preset timer after sending warning information for a first time to the emergency contact person preset in the mobile terminal; and
when the timer expires, sending the warning information for a second time to the emergency contact person preset in the mobile terminal.

20. An electronic device, wherein the electronic device comprises:
at least one processor; and
a memory communicably connected with the at least one processor for storing instructions executable by the at least one processor, wherein execution of the instructions by the at least one processor causes the at least one processor to:
acquire acceleration values from an acceleration sensor at a mobile terminal;
compute whether acceleration suddenly increases and maintains at zero within a preset period after suddenly increasing, according to acquired acceleration values; and
if YES, determine that the body tumble event occurs, and send warning information to an emergency contact person preset in the mobile terminal, wherein the warning information carries current position information.

21. The electronic device according to claim 20, wherein the warning information further comprises current time information.

22. The electronic device according to claim 20, wherein send warning information to an emergency contact person preset in the mobile terminal is: sending warning information to an emergency contact person preset in the mobile terminal by a short message or automatic voice.

23. The electronic device according to claim 21, wherein send warning information to an emergency contact person preset in the mobile terminal is: sending warning information to an emergency contact person preset in the mobile terminal by a short message or automatic voice.

24. The electronic device according to claim 20, wherein compute whether acceleration suddenly increases and maintains at zero within a preset period after suddenly increasing, according to the acquired acceleration values further comprises:
judging, for such two adjacent acceleration values as a current acceleration value and a previous acceleration value, whether a difference value between the two adjacent acceleration values is greater than or equal to a preset value;
if the difference value between the two adjacent acceleration values is greater than or equal to the preset value, judging whether at least two continuous, adjacent acceleration values which are zero exist after the two adjacent acceleration values; and
if at least two continuous, adjacent acceleration values which are zero exist after the two adjacent acceleration values, determining that the acceleration suddenly increases and maintains at zero within a preset period after suddenly increasing.

25. The electronic device according to claim 21, wherein compute whether acceleration suddenly increases and maintains at zero within a preset period after suddenly increasing, according to the acquired acceleration values, comprises:
judging, for such two adjacent acceleration values as a current acceleration value and a previous acceleration value, whether a difference value between the two adjacent acceleration values is greater than or equal to a preset value;
if the difference value between the two adjacent acceleration values is greater than or equal to the preset value, judging whether at least two continuous, adjacent acceleration values which are zero exist after the two adjacent acceleration values; and
if at least two continuous, adjacent acceleration values which are zero exist after the two adjacent acceleration values, determining that the acceleration suddenly increases and maintains at zero within a preset period after suddenly increasing.

26. The electronic device according to claim 20, wherein send warning information to an emergency contact person preset in the mobile terminal is:
starting a preset timer after sending warning information for a first time to the emergency contact person preset in the mobile terminal; and
when the timer expires, sending the warning information for a second time to the emergency contact person preset in the mobile terminal.

27. A non-transitory computer-readable storage medium storing executable instructions that, when executed by an electronic device, cause the electronic device to:
acquire acceleration values from an acceleration sensor at a mobile terminal;
compute whether acceleration suddenly increases and maintains at zero within a preset period after suddenly increasing, according to acquired acceleration values; and
if YES, determine that the body tumble event occurs, and send warning information to an emergency contact person preset in the mobile terminal, wherein the warning information carries current position information.

28. The non-transitory computer-readable storage medium according to claim 27, wherein send warning information to an emergency contact person preset in the mobile terminal is:
sending warning information to an emergency contact person preset in the mobile terminal by a short message or automatic voice.

29. The non-transitory computer-readable storage medium according to claim 27, wherein compute whether acceleration suddenly increases and maintains at zero within a preset period after suddenly increasing, according to the acquired acceleration values, comprises:
judging, for such two adjacent acceleration values as a current acceleration value and a previous acceleration value, whether a difference value between the two adjacent acceleration values is greater than or equal to a preset value;
if the difference value between the two adjacent acceleration values is greater than or equal to the preset value, judging whether at least two continuous, adjacent acceleration values which are zero exist after the two adjacent acceleration values; and
if at least two continuous, adjacent acceleration values which are zero exist after the two adjacent acceleration values, determining that the acceleration suddenly increases and maintains at zero within a preset period after suddenly increasing.

30. The non-transitory computer-readable storage medium according to claim 27, wherein send warning information to an emergency contact person preset in the mobile terminal is:
starting a preset timer after sending warning information for a first time to the emergency contact person preset in the mobile terminal; and
when the timer expires, sending the warning information for a second time to the emergency contact person preset in the mobile terminal.

31. A mobile terminal, comprising:
a positioning module for acquiring geographical position information of the mobile terminal;
an acceleration sensor for collecting acceleration values of the mobile terminal;
a sensor co-processor for acquiring the acceleration values from the acceleration sensor at the mobile terminal, and computing whether acceleration suddenly increases and maintains at zero within a preset period after suddenly increasing, according to acquired acceleration values, and if YES, determining that the body tumble event occurs, and sending a notification message of the body tumble event to an application processor;
the application processor for acquiring current position information from the positioning module upon receipt of the notification message of the body tumble event, and sending warning information carrying the current position information to a modem; and
the modem for sending the warning information to an emergency contact person preset in the mobile terminal.
